(19) United States
(12) Patent Application Publication
(10) Pub. No.: US 2024/0041717 A1
(43) Pub. Date: Feb. 8, 2024

(54) DISINFECTING, SKIN-TOLERABLE, SUPERFATTING CARE AGENT FOR APPLICATION TO THE SKIN

(71) Applicants: TULICHEM GMBH, Mainz (DE); MERLIN APOTHEKE RUTH GEBHARDT UND DR. FRANK ULLRICH OHG, Sankt-Augustin (DE)

(72) Inventors: Rainer Pommersheim, Mainz (DE); Denis Breuch, Mainz (DE); Holger Löwe, Oppenheim (DE)

(21) Appl. No.: 18/017,296

(22) PCT Filed: Jul. 8, 2021
(86) PCT No.: PCT/EP2021/069011

§ 371 Date: Jan. 20, 2023

(30) Foreign Application Priority Data
Jul. 24, 2020 (DE) 20 2020 104 279.3
Aug. 10, 2020 (DE) 202020104615.2

(51) Int. Cl. A61K 8/20; A61Q 17/00; A61Q 19/00; A61K 8/46

(52) U.S. Cl. CPC A61K 8/20; A61Q 17/005; A61Q 19/007; A61K 8/466; A61K 2800/74

(57) ABSTRACT

The invention relates to a disinfecting, skin-tolerable, superfatting care agent for application to the skin or for treatment of the skin, containing one or more disinfecting active substances. According to the invention, water-soluble chlorine derivatives and/or an alcohol are integrated into a base matrix and additionally a substance mixture for masking the odor of the disinfecting active substances is contained.

The invention relates to a disinfecting, skin-tolerable, superfatting care agent for application to the skin and for treatment thereof, containing one or more disinfecting active substances, a lotion based on such an agent, and a dispenser, can, tube or package containing a disinfecting agent or a lotion relating thereto according to the teachings of the independent claims.

Products are known which can be applied to the skin and which, when applied to the skin, have a disinfecting, germicidal effect. Known preparations are based on the active substances isopropanol, ethanol or other alcohols.

Besides liquid products, gels are known in which the alcohol has been thickened.

In order to develop a sufficient disinfecting effect here, alcohol must be present in a concentration of at least 70%. Such a concentration is easily achievable in gels.

A cream or a lotion cannot be manufactured with regard to these concentrations.

Previously known Lotions containing alcohol for disinfection are therefore insufficient in a medical sense and from a hygiene point of view, and their effectiveness is limited. As mentioned, in order to develop a sufficient disinfecting effect, alcohol must be present in a concentration of at least 70% by volume. If it is necessary to add fatty components at this high alcohol content, a wound treatment agent containing chloramine is known from DE 10 2006 043 245 A1.

There, a 10% chloramine-containing wound dressing is obtained by spraying with a chloramine T solution. Together with a physically loaded zeolite component and a liquid-permeable cover material, the presented system may be suitable primarily for bleeding wounds with simultaneous disinfection by the aqueous chloramine solution for care.

In EP 1 955 591 A1, reference is made to a benzalkonium chloride as a disinfectant and antimicrobial skin treatment agent in a cream base with regard to the agent for the care and antimicrobial treatment of the skin. This cream has the aforementioned benzalkonium chloride as its only pharmaceutically active substance. In an ointment in this respect, viscous to solid kerosene, wool wax and/or white vaseline is used as a base. The agent according to EP 1 955 591 A1 is also intended to be suitable for the care disinfection of the skin, in particular for doctors or medical personnel.

From the foregoing, it is the object of the invention to specify a further developed, disinfecting, skin-tolerable, superfatting care agent, in particular a lotion, for application to the skin, which, in addition to good skin compatibility and caring and greasing action, additionally develops a demonstrably sufficient disinfecting action, wherein it is not primarily a question of wound disinfection, but of everyday use for hand disinfection and thus for the interruption of possible chains of infection.

In medical terms, a cream or lotion is an oil-in-water emulsion or water-in-oil emulsion that makes ingredients suitable for application. Creams are less viscous than ointments, i.e. more fluid, and are readily absorbed into the skin. Emulsifiers or emulsion stabilizers are frequently used in creams. In common parlance, lotions are also referred to as non-medicinal, purely skin-care products.

According to the described object, this is solved by a disinfecting, skin-tolerable, superfatting care agent for application to or treatment of the skin according to the combination of features according to claim 1, a lotion according to claim 4 or a dispenser, a can, a tube or a package according to claim 8.

In contrast to known solutions, the agent according to the invention relies on a chlorine derivative as the disinfecting active substance in addition to alcohol. Such an active substance is recommended and suitable as a disinfectant in appropriate concentrations when applied to the skin.

The chlorine derivatives used in the formulation according to the invention are readily soluble in water and can be very easily incorporated into the lotion at the recommended concentrations.

Tests conducted with test persons using the lotion according to the invention prove the germicidal effect with simultaneous good skin tolerance.

Accordingly, the disinfecting, skin-tolerable care agent starts from one or more water-soluble chlorine derivatives, an alcohol or a mixture of said components as the disinfecting active substance.

The disinfecting material or materials are incorporated into a base matrix.

In addition, the lotion contains a mixture of substances to mask the odor of the otherwise olfactory disinfecting materials.

In respect of design, the composition comprises at least one fragrance or fragrance mixture or a thickening agent for viscosity adjustment.

In further development of the invention, a defined amount of functional active substances can be added to the given formulation. These may be, for example, emulsifiers, caring D-panthenol, an active substance for moisturizing or for protection against damaging radiation, in particular ultraviolet radiation.

The lotion according to the invention based on the explained agent is characterized in that the amount of the chlorine derivative used, in particular chloramine-T, is essentially up to 5% by weight, preferably and in particular up to 2% by weight.

The base matrix contains at least glycerides, vaseline and stearates.

The disinfecting ingredient, i.e. the chlorine derivatives, is preferably chloramine-T, sodium hypochlorite or similar substances, as already mentioned.

According to the invention, there is moreover a dispenser, a can, a tube or similar package containing the proposed agent or a corresponding lotion for various applications, in particular for application to human skin.

The invention will be explained in more detail below with reference to an exemplary embodiment.

The base matrix used combines all the ingredients necessary for skin care and ensures the greasing and at the same time caring effect of the lotion.

The disinfecting active substance is based, among other things, on water-soluble chlorine derivatives, which are recognized disinfectants and meet regulatory requirements.

The substance mixture for odor marking is a mixture of dermatologically approved components with the aim of masking the odor released by the disinfecting agent.

Applicable fragrances give the lotion a pleasant smell. These can be essential oils or other fragrances or mixtures made from them.

Among the functionally active substances, urea can be used, for example, to achieve an additional moisturizing effect. However, an agent for absorbing ultraviolet radiation can also be used.

Especially for the base matrix, a commercially available product can be applied.

Such a base matrix includes the following exemplary ingredients:

Glycerol monostearate 60, cetyl alcohol, medium chain triglycerides, white petroleum jelly, macrogol-20-glycerol monostearate, propylene glycol and purified water.

Alcohols, such as ethanol or isopropanol, and/or chlorine derivatives are used for the disinfecting active substance, such as chloramine-T or sodium hypochlorite. In particular, for chloramine-T, the concentration in the cream is 2% by weight. This is a safe upper limit at which a sufficient disinfecting effect can be demonstrated.

Polyacrylates, cellulose derivatives, polyvinylpyrrolidones or copolymers of these compounds are used as thickeners.

Commercially available products can be used for the substance mixture for odor marking.

The following compositions are given by way of example:

Ingredients Mixture A:

2,6-dimethyloct-7-en-2ol, 3,7-dimethyloctan-3-ol, 1-(1,2,3,4,5,6,7,8octahydro-2,3,8,8tetramethyl-2naphthyl)ethan-1-one, benzenepentanol, bmethyl-, isopentyl salicylate, 3-(p-ethylphenyl)-2,2dimethylpropionaldehyde, 3,7-dimethyloctan-3-yl acetate, benzenepropanol, .beta.,.beta.,3-trimethyl-, 2-phenylethanol, anisaldehydes, (E)-4-(2,6,6-trimethyli-cyclohexen-1-yl)-3buten-2-one, oxacyclohexadecen-2-one, piperonal, 1,4-dioxacycloheptadecane5,17-dione, pentadecane-15-olide, hexyl acetate, 3-(o-ethylphenyl)-2,2dimethylpropionaldehyde, 1-methyl-4-(4methylpentyl)cyclohex3-ene-1-carbaldehyde 1;

Ingredients Mixture B:

Hexyl salicylate, 3,7-dimethyloctan-3-ol, 1-(1,2,3,4,5,6,7,8octahydro-2,3,8,8tetramethyl-2naphthyl)ethan-1-one, 2H-pyran-4-ol, tetrahydro-4-methyl-2(2-methylpropyl)-, benzenepentanol, bmethyl-, benzenepropanol, .beta.,.beta.,3-trimethyl-, pentadecane-15-olides, 2,6-dimethyloct-7-en-2ol, α-methyl-1,3benzodioxole-5propionaldehydes, 4-(4-methyl-3pentenyl)cyclohex-3ene-1-carbaldehydes, (E)-anethole, 2-ethyl-4-(2,2,3trimethyl-3-cyclopenten1-yl)-2-buten-1-ol, 1,2,3,5,6,7-hexahydro-1,1,2,3,3-pentamethyl-4H-inden-4-one, 3-(p-ethylphenyl)-2,2dimethylpropionaldehyde, MUGETANOL, piperonal, oxacyclohexadecen-2-one, 4-methyl-3-decen-5-ol, 3-(o-ethylphenyl)-2,2dimethylpropionaldehyde, allyl cyclohexyloxy)acetate, methyl 2,4-dihydroxy-3,6-dimethylbenzoate, 2,6-dimethylhept-5-enal, octahydro-7-methyl-1,4methanonaphthalen6(2H)-one, cis-hex-3-en-1-yl methyl carbonate.

Essential oils, such as rose oil, lavender oil or similar, or mixtures of such essential oils are used as fragrances.

The functional active substances mentioned do not have to be part of the lotion, but can improve its effect and provide for further applications.

As a means of protection against ultraviolet radiation, a titanium dioxide can be added to the lotion to ensure an appropriate sun protection factor.

A ready-to-sell lotion as a disinfecting, skin-tolerable and care agent comprises at least the base matrix and the disinfecting active substance.

The addition of the substance mixture for odor marking makes the application much more acceptable, as the unpleasant chlorine odor of the chlorine derivative(s) used or the disinfecting active substance is subject to encapsulation.

The lotion according to the invention is filled after preparation and production. Filling can be carried out here in a suitable dispenser, can, tube or similar package.

The application of the lotion is carried out by applying it to the skin and gently rubbing it in, which unfolds the disinfecting and at the same time caring effect.

The presented lotion can be used either alone or for subsequent care of the skin.

Known gels, which have a high alcohol content, show problems when fatty or superfatting ingredients are added, as there is a risk of flocculation. According to the invention, this is prevented by adding a thickening agent, preferably polymers and crosspolymers of polycrylates. It proves advantageous that a sequence is followed when stirring in the components.

Another idea according to the invention is to provide encapsulated active substances which are added to the lotion. In this case, the capsules are virtually suspended in the lotion. When applied to the skin, the capsule is destroyed and the active substance contained in the capsule is released. The method of encapsulation and the formation of small beads means that active substances not otherwise absorbed by the lotion can also be added in a targeted and dosed manner.

1. Disinfecting, skin-tolerable, superfatting care agent for application to the skin or treatment of the skin, containing one or more disinfecting active substances, characterized in that water-soluble chlorine derivatives and/or an alcohol are integrated into a base matrix and additionally a substance mixture for masking the odor of the disinfecting active substances is contained.

2. Disinfecting, skin-tolerable care agent according to claim 1, characterized in that it contains at least one fragrance or a fragrance mixture.

3. Disinfecting, skin-tolerable care agent according to claim 1, characterized in that said agent comprises, in a predetermined formulation, functional active substances, for example for moisturizing or for protection against damaging radiation.

4. Lotion based on an agent according to claim 1, characterized in that the amount of chlorine derivatives is substantially up to 5% by weight, in particular up to 2% by weight.

5. Lotion based on an agent according to claim 1, characterized in that the base matrix comprises at least glycerides, vaseline and stearates.

6. Agent or lotion according to claim 1, characterized in that chloramine-T, sodium hypochlorite or a similar chlorine derivative is contained as a disinfecting ingredient.

7. Agent or lotion according to claim 1, characterized in that, with respect to the disinfecting active substance, said substance contains alcohol in a concentration of at least 70% by volume.

8. Dispenser, can, tube or package containing an agent or lotion according to claim 1.
